(19) United States
(12) Patent Application Publication
(10) Pub. No.: US 2026/0250717 A1
(43) Pub. Date: Aug. 27, 2026

(54) A Vaccine Composition of Cells Expressing a Lentiviral Vector and Methods of Using

(71) Applicant: MERIDIAN THERAPEUTICS, INC., Draper, UT (US)

(72) Inventors: Kimberly A. Noonan, Baltimore, MD (US); Ivan R. Borrello, Baltimore, MD (US)

(73) Assignee: MERIDIAN THERAPEUTICS, INC., Draper, UT (US)

(21) Appl. No.: 18/874,959

(22) PCT Filed: Jun. 27, 2023
(86) PCT No.: PCT/US2023/026331

§ 371 Date: Dec. 13, 2024

Related U.S. Application Data
(60) Provisional application No. 63/367,368, filed on Jun. 30, 2022.

(51) Int. Cl. C12N 15/867; A61K 9/00; A61K 31/454; A61K 38/00; A61K 39/00; A61P 35/00; C07K 14/535; C12N 5/09; C12N 15/11

(52) U.S. Cl. CPC C12N 15/867; A61K 9/0019; A61K 31/454; A61K 38/00; A61K 39/0011; A61P 35/00; C07K 14/535; C12N 5/0694; C12N 15/11; A61K 2039/5152; A61K 2039/54; A61K 2039/545; C12N 2510/04; C12N 2740/15043

(57) ABSTRACT

A vector construct is described that is a lentiviral construct including DNA encoding for GM-CSF. A vaccine composition is also described that is includes K562 cells transfected with this vector construct, and also possibly including the U266 and H929. Methods are described for using the vaccine composition in methods of immunizing against plasma cell disorders, including multiple myeloma and related disorders.

### BACKGROUND

### Field of the Invention

The present invention relates to compositions and methods useful for vaccination against plasma cell disorders including multiple myeloma, including a lentiviral vector construct encoding GM-CSF.

### Brief Description of the Related Art

The advent of novel therapeutic agents with fewer toxicities and greater tumor specificity have significantly improved clinical outcomes for patients with multiple myeloma (MM). Specifically, triple therapy with a proteasome inhibitor, an immunomodulatory derivative (IMiD), and steroids generate increased response rates and more durable remissions (Attal et al.; N. Engl. J. Med. (2017) 376:1311-20; Durie et al.; Lancet (2017) 389:519-27). This has translated into improved progression-free survival (PFS) and overall survival (OS) (Richardson et al.; Hematology (2014) 255-61).

However, a significant proportion of patients will eventually develop resistance to these agents and relapse. Approaches aimed at deepening and prolonging these responses have included long-term maintenance therapies with or without consolidation regimens. In particular, lenalidomide (Len) maintenance in both the transplant and non-transplant settings has shown the significant clinical benefit of such approaches (McCarthy et al.; J Clin Oncol (2017) 35:3279-89).

With the introduction of more effective treatments for MM, much of the overall improvement in clinical outcomes is likely due to the ability to achieve deeper responses. In fact, minimal residual disease (MRD) assessment by either flow cytometry or next generation sequencing can now detect as few as one in a million cells (ClonoSEQ Cleared for Residual Cancer Testing. Cancer Discov (2018) 8: OF6-OF6).

A growing body of literature strongly correlates the depth of response with improved clinical outcome to the point that MRD negativity is now being considered as a potential approvable endpoint for clinical trials (Perrot et al.; Blood (2018) 132:2456-64). However, definitive clinical guidance and safe treatment options for the management of MRD-positive MM patients is much needed in the art.

Immunotherapy exploits the capacity of the immune system to specifically recognize and eliminate cancer cells. In fact, immune checkpoint blockade (Hargadon et al.; Int. Immunopharmacol. (2018) 62:29-39) and genetically engineered T cells bearing chimeric antigen receptors (CAR-T) (Raje et al.; N. Engl. J. Med. (2019) 380:1726-37) have demonstrated clinical efficacy in hematologic malignancies and, on a more limited basis, in solid tumors. Conversely, cancer vaccines to date have not shown the same benefits (Hu et al.; Nat. Rev. Immunol. (2018) 18:168-82).

The generation of productive vaccine-specific immune responses depends on the diversity and abundance of tumor-associated antigens, an effective adjuvant and concomitant immunostimulatory therapy. The established cell line K562 has been genetically modified to produce granulocyte-macrophage colony-stimulating factor (GM-CSF). GM-CSF is a key immunostimulatory factor shown to improve efficient antigen presentation (Borrello et al.; Hum. Gene Ther. (1999) 10:1983-91; Borrello et al.; Cytokine Growth Factor Rev. (2002) 13:185-93).

Although several factors, such as poor tumor antigen selection, choice of vaccine adjuvants and the absence of concomitant immunomodulatory therapy may account for the lack of clinical efficacy, the disease burden is likely to have a relevant impact on clinical outcomes. Although the ability of lenalidomide to augment vaccine-specific cellular and humoral immunity has been demonstrated (Noonan et al.; Clin. Cancer Res. (2012) 18:1426-34), for MRD-positive multiple myeloma, which implies a very low disease burden, multiple myeloma vaccination in combination with lenalidomide has never been shown to be a possible therapeutic approach to enhance treatment efficacy without additional toxicity. Effective therapies for the treatment of MRD positive MM and other plasma cell disorders are currently lacking and no consensus on the management of this ever-growing patient population exists. Described herein is the first study of successfully treating patients with a minimal disease burden to improve the disease response as well as to prevent disease progression using a vaccine composition that includes a newly designed lentiviral vector.

### SUMMARY

According to a first aspect of the invention, a composition is described for use in raising an immune response to a plasma cell disorder in a subject comprising an effective amount of U266, H929, and K562 cells.

According to another aspect of the invention, a lentiviral vector construct is described comprising a DNA sequence encoding GM-CSF.

According to another aspect of the invention, the lentiviral vector construct as described above is described, further comprising a DNA sequence encoding EF1a.

According to another aspect of the invention, the lentiviral vector construct as described above is described, wherein the DNA sequence encoding EF1a is shown in SEQ ID NO: 2.

According to another aspect of the invention, the lentiviral vector construct as described above is described, wherein an amino acid sequence of GM-CSF is shown in SEQ ID NO: 3.

According to another aspect of the invention, the lentiviral vector construct as described above is described, comprising the structure LTR-EF1a-GMCSF-LTR.

According to another aspect of the invention, the lentiviral vector construct as described above is described, wherein DNA encoding the LTR is shown in SEQ ID NO: 1.

According to another aspect of the invention, the lentiviral vector construct as described above is described, wherein the K562 cells comprise the vector construct as described above.

According to another aspect of the invention, the lentiviral vector construct as described above is described, wherein the composition is a vaccine.

According to another aspect of the invention, the lentiviral vector construct as described above is described, wherein said vaccine is allogeneic.

According to another aspect of the invention, the lentiviral vector construct as described above is described, further comprising U266 and H929 cells.

According to another aspect of the invention, the lentiviral vector construct as described above is described, wherein the DNA sequence encoding GM-CSF is able to produce GM-CSF in an amount of up to about 1500 ng/1×10⁶ cells.

According to another aspect of the present invention, the composition as described above is provided, wherein the composition is a vaccine.

According to another aspect of the present invention, the composition as described above is provided, wherein said vaccine is allogeneic.

According to another aspect of the present invention, the composition as described above is provided, wherein the K562 cells express GM-CSF.

According to another aspect of the present invention, the composition as described above is provided, wherein the K562 cells have been transfected with a vector construct encoding GM-CSF.

According to another aspect of the present invention, the composition as described above is provided, wherein the gene encoding GM-CSF is able to produce GM-CSF in an amount of up to about 1500 ng/1×10⁶ cells.

According to another aspect of the present invention, the composition as described above is provided, wherein the amount of GM-CSF produced is between about 35-1200 ng/1×10⁶ cells.

According to another aspect of the present invention, the composition as described above is provided, wherein the GM-CSF is derived from human.

According to another aspect of the present invention, the composition as described above is provided, wherein the ratio of the combination of U266 and H929 cells to K562 cells is about 20:1.

According to another aspect of the present invention, the composition as described above is provided, wherein the U266 and H929 cells are present in equal amounts.

According to another aspect of the present invention, the composition as described above is provided, wherein the U266 and H929 cells are present in unequal amounts

According to another aspect of the present invention, the composition as described above is provide, wherein said composition induces an immune response in the subject when administered to said subject.

According to another aspect of the present invention, the composition as described above is provided, wherein the immune response induces complete remission of said plasma cell disorder in the subject.

According to another aspect of the present invention, the composition as described above is provided, wherein the composition prolongs progression free survival in said subject.

According to another aspect of the present invention, the composition as described above is provided, wherein said complete remission is determined as a non-detectable M-spike and positive immunofixation electrophoresis.

According to another aspect of the present invention, the composition as described above is provided, wherein the subject is a human.

According to another aspect of the present invention, a method of inducing complete remission in a subject having multiple myeloma is provided, comprising administering to the subject the composition as described above.

According to another aspect of the present invention, the method as described above is provided, wherein said administering comprises also giving lenalidomide to said subject.

According to another aspect of the present invention, the method as described above is provided, wherein said lenalidomide is given to said subject before, during, and/or after said administering.

According to another aspect of the present invention, the method as described above is provided, wherein the composition is a vaccine.

According to another aspect of the present invention, the method as described above is provided, wherein the vaccine is allogeneic.

According to another aspect of the present invention, the method as described above is provided, wherein the K562 cells express a GM-CSF gene.

According to another aspect of the present invention, the method as described above is provided, wherein the K562 cells have been transfected with a gene encoding GM-CSF.

According to another aspect of the present invention, the method as described above is provided, wherein the GM-CSF gene is able to express an amount of GM-CSF of up to about 1500 ng/1×10⁶ cells.

According to another aspect of the present invention, the method as described above is provided, wherein the GM-CSF gene is able to express an amount of GM-CSF of about 35-1200 ng/1×10⁶ cells.

According to another aspect of the present invention, the method as described above is provided, wherein the amount of GM-CSF is produced, on average, every 24 hours.

According to another aspect of the present invention, the method as described above is provided, wherein the GM-CSF is derived from human.

According to another aspect of the present invention, the method as described above is provided, wherein the ratio of the combination of U266 and H929 cells to K562 cells is about 20:1.

According to another aspect of the present invention, the method as described above is provided, wherein the dose of said composition is such that the ratio of tumor cells in said subject to K562 cells in said composition is greater than 2:1.

According to another aspect of the present invention, the method as described above is provided, wherein the U266 and H929 cells are present in equal amounts in said composition.

According to another aspect of the present invention, the method as described above is provided, wherein said U266 and H929 cells are present in said composition in an amount of about 5×10⁷ cells and the K562 cells are present in said composition in an amount of about 5×10⁶ cells.

According to another aspect of the present invention, the method as described above is provided, wherein said plasma cell disorder is selected from the group consisting of MGUS, SMM, multiple myeloma, non-secretory multiple myeloma, indolent myeloma, light chain myeloma, plasma cell leukemia, and primary amyloidosis.

According to another aspect of the present invention, the method as described above is provided, wherein said plasma cell disorder is multiple myeloma.

According to another aspect of the present invention, the method as described above is provided, wherein said complete remission persists in said subject for up to 5 years.

According to another aspect of the present invention, the method as described above is provided, wherein said complete remission is determined by measuring no detectable monoclonal spike and negative immunofixation electrophoresis.

According to another aspect of the present invention, the method as described above is provided, wherein said subject is positive for minimal residual disease.

According to another aspect of the present invention, the method as described above is provided, wherein said composition minimizes a non-specific immune response in the subject.

According to another aspect of the present invention, the method as described above is provided, wherein said composition is administered to said subject in 1 to 5 doses, spaced apart by more than 1 day between each dose.

According to another aspect of the present invention, the method as described above is provided, wherein 2 to 4 doses are administered, spaced apart by more than 2 weeks between each dose.

According to another aspect of the present invention, the method as described above is provided, wherein 2 to 4 doses are administered, spaced apart by more than 4 weeks between each dose.

According to another aspect of the present invention, the method as described above is provided, wherein 4 doses are administered, spaced apart by about 1 month between each dose.

According to another aspect of the present invention, the method as described above is provided, wherein the first 3 doses are spaced apart equidistantly.

According to another aspect of the present invention, the method as described above is provided, wherein all doses are administered within one year relative to each other.

According to another aspect of the present invention, the method as described above is provided, wherein at least one dose is administered between and including days 7-18 relative to starting a course of lenalidomide.

According to another aspect of the present invention, the method as described above is provided, wherein at least one dose is administered on about day 15 relative to starting a course of lenalidomide.

According to another aspect of the present invention, a method of prolonging progression free survival in a subject having multiple myeloma is provided comprising administering the composition of claim 1 to said subject in combination with lenalidomide.

According to another aspect of the present invention, the method as described above is provided, a method of inducing an increase in clonal T-cell expansion and a myeloma-specific cytokine response in a subject having multiple myeloma is provided comprising administering to the subject the composition of claim 1 in combination with lenalidomide.

According to another aspect of the present invention, the method as described above is provided, wherein said increase persists in said subject for up to 7 years after said administering.

According to another aspect of the present invention, the method as described above is provided, wherein said increase persists in said subject for up to 5 years after said administering.

According to another aspect of the present invention, a method of inducing multiple-myeloma-specific immunity in a subject is provided comprising administering to the subject the composition of claim 1 in combination with lenalidomide.

According to another aspect of the present invention, the method as described above is provided, wherein said subject is positive for minimal residual disease at the time of said administering.

According to another aspect of the present invention, a method of preventing relapse of multiple myeloma in a subject is provided, comprising administering to the subject the composition of claim 1 in combination with lenalidomide.

According to another aspect of the present invention, the method as described above is provided, wherein the subject is positive for minimal residual disease at the time of said administering.

According to another aspect of the present invention, the method as described above is provided, wherein the subject is a human.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the compositions and methods, given only by way of example, and with reference to the accompanying drawings, in which:

FIG. 1 illustrates the lentiviral vector construct, wherein LTR are long terminal repeat sequences, EF1a is a promoter sequence, and GM-CSF is granulocyte-macrophage colony-stimulating factor.

FIG. 2 shows the sequence of the LTRs (SEQ ID NO: 1), which flank either side of the EF1a promoter and the GM-CSF coding sequence.

FIG. 3 shows the sequence of the EF1a promoter (SEQ ID NO: 2)

FIG. 4 shows the amino acid sequence of the GM-CSF protein product (SEQ ID NO: 3).

FIG. 5 shows a scheme of the clinical trial. Patients received four doses of vaccine at the indicated timepoints (arrows) while on Len maintenance. “*” indicates immune monitoring timepoints.

FIG. 6 shows the frequency of T-cell clones expanded at C3D14 tracked over time in blood and bone marrow in all patients.

FIG. 7 shows representative pairwise scatterplots of two patients showing clonal expansion of pre-existing T-cell clones after vaccination as well as the recruitment of novel clonotypes previously absent in either PB or BM.

FIG. 8 shows representative pairwise scatterplots comparing the fold change in the frequency of expanded T-cell clones in PB and BM.

FIG. 9 shows data representing changes in the Morisita Index, which quantifies the degree of similarity between the BM and PB T-cell repertoires, before, during (C3D14), and after vaccination. TCR=T cell receptor.

FIG. 10 shows representative plots showing IFNγ and TNFα production before, during (C3D14), and after vaccination in both CD8⁺ and CD4⁺ T cell compartments.

FIG. 11 shows cytokine production increased after vaccination in all patients and was maintained for more than 4 years (p<0.0001 for both CD8⁺ and CD4⁺compartments).

FIG. 12 shows boxplots showing frequencies of each individual cluster across patients and timepoints.

FIG. 13 shows T cell clones expanded post-vaccination tracked in both PB and BM up to 7 years after MM-GVAX administration.

FIG. 14 shows representative plots showing IFNγ and TNFα production upon in vitro antigen-stimulation of BM from vaccinated patients at the indicated, long-term follow-up timepoints. *, p<0.05; **, p<0.01; ***, p<0.001; ****, p<0.0001.

FIG. 15 shows that the frequency of CD69+ T cells is significantly higher in the CD8+subset (p<0.001).

FIG. 16 shows representative dot plots and histograms showing the canonical phenotype of CD69+BM T cells.

FIG. 17 shows representative histograms depicting expression of different markers on CD69⁺ (red) and CD69⁻ (light blue) BM T cells. *, p<0.05; **, p<0.01; ***, p<0.001; ****, p<0.0001.

FIG. 18 shows boxplots representing relative abundance of the 8 FlowSOM metaclusters in the two groups (relapse and responder).

FIG. 19 shows representative dot plots showing manual gating analysis of DNAM1^{-/low} CD27⁻ CD8⁺ T cells (left) and summary of the frequency of this CD8⁺ T cell subset in both groups. *, p<0.05; **, p<0.01; ***, p<0.001; ****, p<0.0001.

### DETAILED DESCRIPTION OF EXEMPLARY EMBODIMENTS

A lentiviral vector construct encoding GM-CSF is described herein that can be used to transfect K562 cells. The vector construct includes 2 LTR sequences flanking a promoter sequence and the target protein to be expressed, which can be GM-CSF. The K562 cells transfected with the lentiviral vector construct can be used to produce an allogeneic whole-cell GM-CSF-secreting multiple myeloma (MM) vaccine. Further described herein are methods of administering this vaccine in combination with lenalidomide, sometimes referred to as “Len”, in MM patients with a minimal residual disease burden defined as no detectable monoclonal spike but positive immunofixation electrophoresis (IFE), demonstrating eradication of residual disease and conversion to complete remission (CR). The vaccine is likely to also be effective against other plasma cell disorders that are characterized by elevated monoclonal spike protein. Safety, time to response, and immune monitoring of vaccine- and MM-specific T cell responses are also described. To our knowledge, this is the first study attempting to treat patients with a minimal disease burden in an effort to further improve the disease response as well as to prevent disease progression using a novel lentiviral vector construct encoding GM-CSF.

As used herein and unless otherwise indicated, the term “about” is intended to mean±5% of the value it modifies. Thus, “about 100” means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “treat,” “treated,” or “treating” mean both therapeutic treatments wherein the object is to slow down (lessen) an undesired physiological condition, disorder, or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” or treatment of “multiple myeloma” or treating “multiple myeloma” means an activity that alleviates or ameliorates any of the primary phenomena or secondary symptoms or presentations associated with the cancer, multiple myeloma, or any other condition described herein. In some embodiments, the cancer that is being treated is one of the cancers recited herein. In one embodiment, the cancer is multiple myeloma.

As used herein, the term “subject” can be used interchangeably with the term “patient”. The subject can be a mammal, such as a dog, cat, monkey, horse, or cow, for example. In some embodiments, the subject is a human. In some embodiments, the subject has been diagnosed with a hematological cancer. In some embodiments, the subject has been diagnosed with multiple myeloma. In some embodiments, the subject is suspected of having multiple myeloma.

As used herein, the term “express” as it refers to a cell surface receptor, such as, but not limited to, CD3, CD4, and CD8, can also be referred to as the cell being positive for that marker. For example, a cell that expresses CD3 can also be referred to as CD3 positive (CD3+).

As used herein, the term “express” can also refer to gene located within the cell, either as a part of the chromosomal DNA, or on some other vector. A cell “expresses” a gene when that gene is induced to produce the protein that it encodes. The produced protein can either be harbored within the cell or transported outside of the cell.

As used herein, the term “vector construct” relates to a DNA or RNA molecule, such as a plasmid or virus, that can be used as a vehicle to carry a particular DNA segment into a host cell as part of a cloning or recombinant DNA technique. The lentiviral vector construct as described herein can assist in replicating and/or expressing the inserted target DNA sequence constitutively, which enable improved expression and production of the target DNA as compared to simple plasmids, particularly those tied to selective expression using antibiotic or other sorts of markers.

As used herein, the term “vaccine” refers to a product or composition that stimulates a subject's immune system to produce immunity to a specific disease or condition, thus protecting the subject from that disease or condition. The vaccine may be a part of a composition and the composition may or may not contain other components, including but not limited to adjuvants.

As used herein, the term “adjuvant” refers to an ingredient that modifies the action of a principal ingredient, such as a vaccine. An adjuvant when used in a vaccine composition, can help to create a stronger immune response in the subject receiving the vaccine composition.

The term “cancer” as used herein is defined as disease characterized by the rapid and uncontrolled growth of aberrant cells. Cancer cells can spread locally or through the bloodstream and lymphatic system to other parts of the body.

The term “multiple myeloma” as used herein is defined as cancer originating in the white blood cells. In some embodiments, the white blood cells are in the bone marrow. In some embodiments, the multiple myeloma originates in the plasma cells.

The term “plasma cell disorder” as used herein is that as defined by the International Myeloma Working Group, including blood cancers in which plasma cells become malignant and infiltrate the bone marrow.

“Effective amount” or “therapeutically effective amount” are used interchangeably herein, and refer to an amount of a compound, formulation, material, or composition, as described herein effective to achieve a particular biological result. Such results may include, but are not limited to, the inhibition of cancer cell proliferation as determined by any means suitable in the art.

As used herein, GM-CSF refers to granulocyte-macrophage colony-stimulating factor, which is a known protein often used in cancer treatments. The original work with GM-CSF, also called “Leukine” in the literature, involved proliferative stimulation of macrophages and neutrophils for the purpose of reducing hematopoietic toxicity related to dose-intensive chemotherapy. The GM-CSF gene when transfected into tumor cells and administered as a vaccine has demonstrated tumor regression and prolonged survival in both animal models and early clinical trials. (Nemunaitis, Expert Rev Vaccines; 2005; 4 (3): 259-74).

As used herein, the term “GVAX” refers to a cancer vaccine composed of whole tumor cells genetically modified to secrete the immune stimulatory cytokine GM-CSF. One or more cell types can be included in a GVAX vaccine.

As used herein, the term “lenalidomide”, also known by its trade name Revlimid, is a medication used to treat multiple myeloma and myelodysplastic syndromes (MDS). It can be administered with steroids, including but not limited to dexamethasone.

As used herein, the term “minimal residual disease” or “MRD” refers to the small number of cancer cells that remain in the body after treatment. The number of remaining cells may be so small that they do not cause any physical signs or symptoms and often cannot even be detected through traditional methods, such as viewing cells under a microscope and/or by tracking abnormal serum proteins in the blood. An MRD positive test results means that residual (remaining) disease was detected. A negative result means that residual disease was not detected. As described herein, MRD is used to measure the effectiveness of treatment and to predict which patients are at risk of relapse. When a patient tests positive for MRD, it means that there are still residual cancer cells in the body after treatment. When MRD is detected, this is known as “MRD positivity.” When a patient tests negative, no residual cancer cells were found. When no MRD is detected, this is known as “MRD negativity.”

The subjects who are candidates for the administration of the composition vaccine as described herein can also have received or currently be receiving immunomodulatory drugs, including but not limited to, thalidomide, lenalidomide and pomalidomide, and proteasome inhibitors, including but not limited to bortezomib, carlfilzomib and ixazomib. The subjects who are candidates for the administration of the composition vaccine as described herein can have a plasma cell disorder. Subjects with plasma cell disorders can be identified by elevated serum levels of M spike protein, or “M-spike”, but this is not a required condition. The subjects with plasma cell disorders include but are not limited to those diagnosed with monoclonal gammopathy of undetermined significance (“MGUS”); multiple myeloma (“MM”), including smoldering myeloma (“SMM”), non-secretory multiple myeloma, indolent myeloma, and light chain myeloma; plasma cell leukemia, and primary amyloidosis.

Next generation sequencing (NGS) techniques as well as flow cytometry have enabled the quantification of the “minimal residual disease” (MRD) burden in patients with MM. MRD negativity is a major prognostic factor in MM and is increasingly becoming a measurable endpoint of studies as well as treatment as it is now more achievable. However, guidance on how to optimally treat MM patients in complete remission (CR), but still with MRD positivity is currently lacking. The vaccine composition as described herein can generate a specific immune response against MM plasma cells eradicating a small residual disease burden which translates into clinically meaningful outcomes.

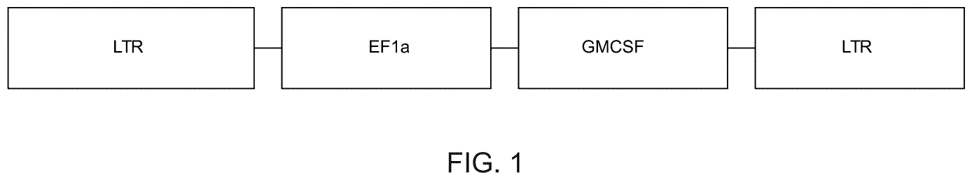

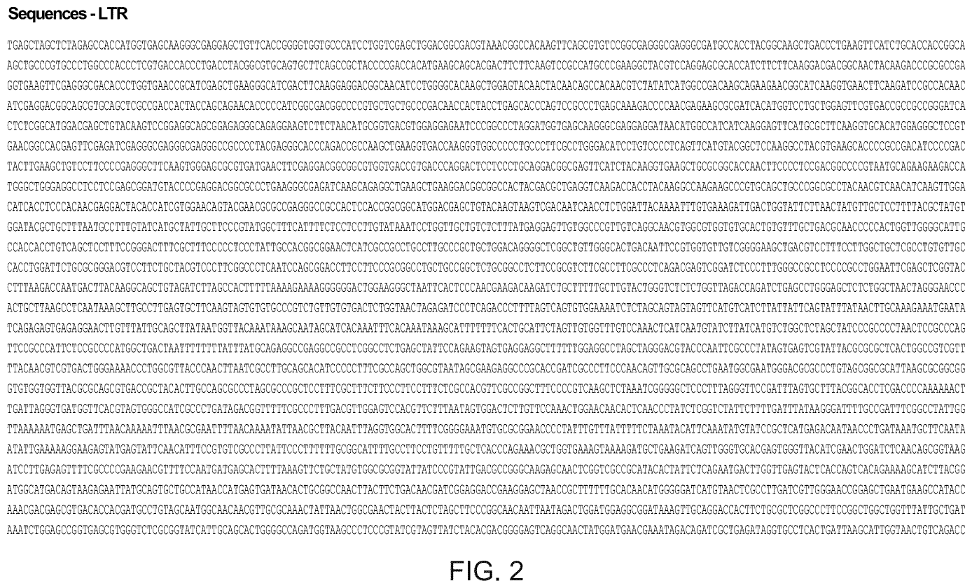

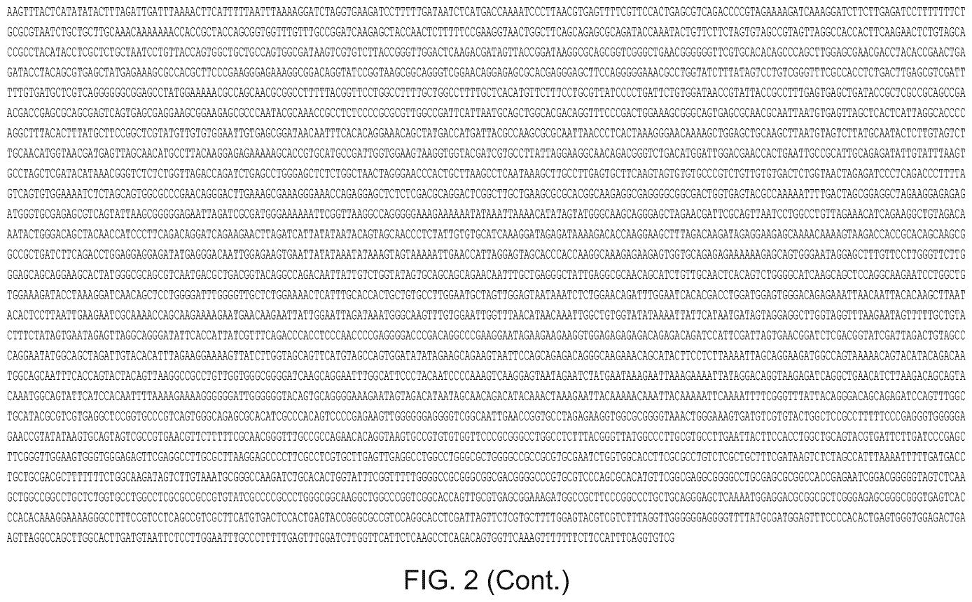

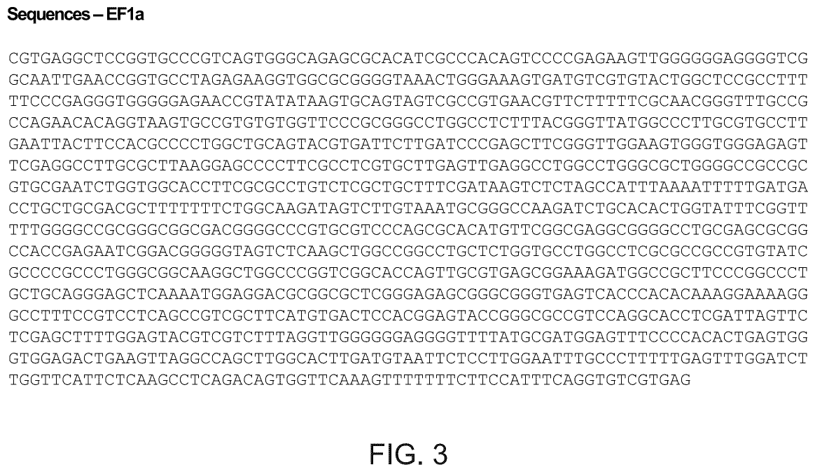

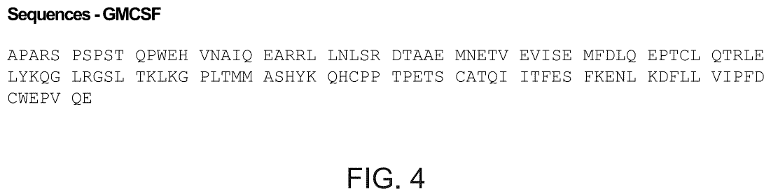

The vector construct can have the composition as shown in FIG. 1, but is not limited to this composition. A promoter that can be used to aid in expression of the GM-CSF protein is the EF1a promoter (SEQ ID NO: 2; FIG. 3), but any promoter that can increase the constitutive expression of the GM-CSF protein in the chosen host cell can be used. The vector construct can also include one or more long terminal repeats (“LTR”) (SEQ ID NO: 1; FIG. 2). A LTR is typically included in the vector construct as a pair of identical DNA sequences that when transcribed with a target sequence, such as GM-CSF (SEQ ID NO: 3; FIG. 4), can enable constitutive expression and integration of the target sequence into the host genome or chromosome.

The allogeneic GM-CSF-producing MM vaccine (MM-GVAX) as described herein can include 3 or more distinct cell lines, including but not limited to the known heterologous MM cell lines, H929 and U266, both publicly available from cell line depositories such as ATCC (Manassas, VA; ATCC.org), as well as K562 cells, also publicly available. The K562 cell line can be transfected or transformed with the vector construct as described herein, that includes a gene encoding GM-CSF in such a configuration so that it can be expressed. Promoter sequences and other know vector components such as long terminal repeats (“LTR”) can also make up the vector construct. Expression constructs that can be used include those that include typical known components such as those that enable optimum expression in the host cell, such as a promoter, operator, origin of replication, and the like, operably linked to the GM-CSF coding sequence (see FIG. 1).

The amounts of cells of each cell line within the vaccine composition is not limited and can be equal or unequal amounts of each cell line, relative to each other. The ratio of the number of one kind of cell line to another can be equal, but is not limited to this ratio. The ratio of H929 and U266 can be 1:1, but is not limited to this ratio, and can also be present in unequal amounts. The ratio of the amount of combined H929/U266 cells to K562/GM-CSF can be about 40:1 to K562/GM-CSF, or can be about 35:1, 30:1, 25:1, 20:1, 15:1, or 10:1. One embodiment is a ratio of about 20:1. Regardless of the ratios of the cell lines, one embodiment is that there is about of 50-1500 ng/1×10⁶cells/24 hours of GM-CSF.

The absolute amounts of the cells present in the vaccine can be about 1×10⁷ to about 1×10⁹ for each of the H929 and U266 cells, and including all amounts in between 1, 5, 10, 50, or 100×10⁷. An embodiment includes wherein the composition has equal amounts of 5×10⁷ cells of each of H929 and U266. The K562/GM-CSF cells can be present in an amount from about 1×10⁴ to about 1×10⁷, including all amounts in between 1, 5, 10, 50, or 100×10⁷. An embodiment includes wherein the composition has an amount of K562/GF-CSF cells of 1×10⁶.

The vaccine composition can contain ingredients other than the 3 or more cell lines, including but not limited to other cell lines, adjuvants such as aluminum, such as aluminum hydroxide, aluminum phosphate, and potassium aluminum sulphate; squalene oil such as MF59; preservatives such as thiomersal or thimerosal; a stabilizer such as Gelatine, sorbitol, sucrose, lactose, mannitol, glycerol, medium 199, arginine hydrochloride, monosodium glutamate, and urea; and emulsifiers, such as polyforbate 80, sorbitan trioleate, and sodium citrate. Other ingredients commonly used in vaccine manufacture can be present and can include antibiotics, ovalbumin, yeast proteins, latex, formaldehyde, glutaraldehyde; and regulators, such as acidity regulators, such as salts based on sodium and/or potassium, disodium adipate, succinic acid, sodium hydroxide, histidine, sodium borate, trometamol, and human serum albumin. These ingredients can be included in amounts that are typical in vaccine formulation, and can be any amount, although typically are very small, as long as the presence of the ingredient does not negatively affect the effectiveness of the vaccine. Human serum albumin is typically used at between 0 and 10%.

The allogeneic GM-CSF-producing MM vaccine (MM-GVAX) as described herein can be administered to subjects with a diagnosis of a plasma cell disorder, for example, multiple myeloma (MM). Candidate MM patients can have a positive or negative MRD. Candidate MM patients can have a low disease burden. Candidate MM patients can have achieved a stable near CR (nCR), defined as an absent M-spike and a positive IFE in either serum or urine, for at least 4 months. The rate of conversion from nCR to true CR was 53.3% with 8 patients improving their clinical response within a median time of 11.6 months from enrollment.

The mode of administration of the vaccine composition as described herein is not particularly limited, and can include an oral route, a subcutaneous route, an intramuscular route, an intradermal route, and an intranasal route. The vaccine composition can be administered one time, 2 times, 3 times, 4 times, or 5 or more times. The amount of time in between administrations of the vaccine composition as described herein is not limited and can be any amount between 1 week and 4 months between administrations, such as 2 weeks, 3 weeks, 4 weeks, 1 month, 2 months, 3 months, and any time amount in between these. The time between multiple doses does not have to be the same. A particular example is 1 month between vaccine administrations. An example of an administration schedule is that all vaccine doses are given within 1 year, 11 months, 10, months, 9 months, 8 months, 7 months, 6 months, or 5 or less months, including all time points in between.

Several factors impact the generation of active, tumor-specific T cells as a result of vaccine administration; specifically, the presence of appropriate antigens, effective antigen presentation, inhibition of the suppressive tumor microenvironment and expression of the appropriate chemokines all facilitate efficient T cell trafficking to the tumor site. The vaccine composition as described herein takes into account several of these key components.

First, the vaccine composition as described herein serves as a source of tumor-associated antigens (TAA) due to the presence of the two established heterologous MM cell lines, H929 and U266. These cell lines display a diverse pattern of somatic mutations frequently associated with high risk and relapsed MM. Specifically, H929 harbors a t (4; 14) translocation and a mutated NRAS, while U266 has several mutations involving the BRAF and TP53 pathways (Moreaux et al.; Haematologica; 2011; 96:574-82). Disease relapse is known to sometimes occur as a result of clonal evolution leading to more aggressive genetic mutations. The vaccine composition as described herein has been designed to prime the immune system to several of these putative high-risk antigens prior to their appearance in the process of clonal evolution associated with disease progression. This presentation of these high-risk antigens via the vaccine composition as described herein is shown to significantly impact the timing and/or aggressiveness of disease relapse.

Second, the vaccine composition as described herein can include, along with the two unmodified MM cell lines H929 and U266, a genetically modified bystander GM-CSF-secreting cell line, K562/GM-CSF, which contains a lentiviral vector construction as described herein. The GM-CSF gene used in the lentiviral vector that is used to transfect the K562 cells can be derived from any source, including but not limited to human. “Derived from” as used herein can mean native to, that is, how or where the GM-CSF exists in nature.

GM-CSF has been shown to be a key immune adjuvant. Importantly, the use of the K562/GM-CSF cell line allows for the titering of the amount of GM-CSF so to deliver the optimal dose within the vaccine composition as described herein. This dose of GM-CSF can be neither insufficient nor supratherapeutic so to reduce its efficacy through the induction of myeloid derived suppressor cells (MDSCs) while still delivering a high dose of antigen. It has been shown that an effective vaccine requires a “therapeutic” dose of GM-CSF and sufficient amount of antigen. (Serafini et al.; Cancer Res. 2004; 64:6337-43). As described herein, the K562 cells can express the GM-CSF as a part of the lentiviral vector construct in an amount of about 50 ng to about 1500 ng per 1×10⁶ cells. This amount can be produced over a period of time or all at once. The period of time over which the GM-CSF can be produced can be up to about 72 hours as measured by ELISA, but can be more or less, as necessary to maintain an effective amount of the vaccine composition. The amount as described above can be produced on average, every 24 hours. It also requires that the antigen cell source, that is the tumor cell, be present in excess so that the stoichiometry of tumor cell: bystander cell is at least greater than 2:1. The amount of GM-CSF can be measured by any known method, including but not limited to enzyme-linked immunosorbent assay (“ELISA”).

The vaccine composition can be irradiated using known methods, which may inhibit proliferation of the tumor cell lines and induce immunogenic cell death to improve antigen delivery. The dose of the vaccine is typically in a ratio relative to the tumor cells of 2:1, particularly that the ratio of tumor cells to K562/GM-CSF cells is 2:1. Determination of the amount of tumor cells can be determined by known methods, including but not limited to flow cytometry.

Third, immunomodulatory drugs (IMiDs), including but not limited to lenalidomide, can markedly improve T cell responses in cancer patients and enhance vaccine efficacy of the vaccine composition as described herein. The IMiDs that can be administered with the vaccine composition as described herein include but are not limited to lenalidomide, thalidomide, and pomalidomide. Lenalidomide is a particular example.

Lenalidomide (sometimes called “Len” in the literature) can be used as a vaccine adjuvant or can be co-administered with the vaccine composition in the methods as described herein. The lenalidomide can be administered at any time prior to administration of the vaccine composition, can be co-administered with the vaccine composition, or can be administered after the vaccine composition. The dose of lenalidomide can range from 2.5-25 mg/per dose. The amount of time before and after the administration of the vaccine composition is not limited and includes up to 10 years either before or after, can be up to 4 years before or after, can be 3 years before or after, can be 2 years before or after, or can be 1 year before or after, and any time points in between these time points, including but not limited to 12, 11, 10, 9, 8, 7, 6, 5, 4, 3, 2, and 1 months before or after. The administration of lenalidomide can be continuous or in several separate administrations. Administration of the vaccine composition as described herein, in combination with continuous lenalidomide administration and a low tumor burden is shown to provide effective, long-lasting anti-MM immunity.

Expansion and persistence of T cell clonotypes by bulk TCR repertoire analysis can be observed in both blood and bone marrow (BM) following administration of the vaccine composition as described herein. Hence, both local expansion of MM-specific clones induced by the vaccine composition as described herein in the bone marrow, and possibly infiltration from peripherally derived clonotypes, can cause the development and maintenance of a durable immune response. Furthermore, the increase in polyfunctional cytokine-producing T cells in response to stimulation with both the vaccine composition-related antigens and non-vaccine-related MM-antigens support the development of broad immune responsiveness towards a diverse set of heterologous, shared MM-antigens. Hence, the allogeneic, whole-cell vaccine as described herein overcomes the barriers imposed by patient-specific tumor heterogeneity and can elicit effective T cell responses against shared, tumor specific antigens while imparting long-lasting antitumor immunity.

The vaccine composition as described herein is shown to promote the ability to detect T cell clonotypes that expanded post-vaccination and characterize the polyfunctional cytokine T cell responses for up to seven years after vaccination.

Hence, the vaccine composition as described herein enables reversion to and maintenance of a myeloma-monoclonal-gammopathy-of-undetermined-significance state, known as “MGUS”, which is an early stage of multiple myeloma and is actually not cancer at all. MGUS is a benign condition indicated by a low level of M-protein, a low level of abnormal plasma cells in bone marrow, and no indicators of active disease. This status can be held in check by continued activity of T cell-mediated immunity induced by the vaccine composition as described herein, and is identified by the presence of a tissue resident-like CD8⁺ T cell population in the bone marrow of these patients. Hence, patients who have been diagnosed with MGUS, but have not progressed to multiple myeloma, are also candidates for the vaccine as described herein. Maintenance of a patient in MGUS via the administration of the vaccine as described herein can enable prevention of progression to myeloma.

Complete remission in a patient with multiple myeloma, as measured by the patient having no detectable M-protein spike and a negative measurement in immunofixation electrophoresis, can be achieved by administering the vaccine composition by the methods and dosage schedules as described herein, and therefore, methods of inducing a complete remission is these patients are possible. The complete remission can persist in the patient for up to 5 years, up to 6 years, or up to 7 years.

Prolonging progression free survival in a subject having multiple myeloma, as measured by determining the time of diagnosis until the date of progression, relapse or relapse, can be achieved by administering the vaccine composition by the methods and dosage schedules as described herein, and therefore, methods of prolonging progression free survival is these patients are possible. Progression free survival can be measured for up to 5 years, 6 years, or up to 7 years.

Increasing clonal T-cell expansion and a myeloma-specific cytokine response in a patient with multiple myeloma can be achieved by administering the vaccine composition by the methods and dosage schedules as described herein, and therefore, methods of increasing clonal T-cell expansion and a myeloma-specific cytokine response in these patients are possible.

Inducing multiple-myeloma-specific immunity in a patient with multiple myeloma can be achieved by administering the vaccine composition by the methods and dosage schedules as described herein, and therefore, methods of inducing multiple-myeloma-specific immunity in these patients are possible.

Preventing relapse of multiple myeloma in a patient who had previously had a positive diagnosis of multiple myeloma but had previously achieved negative MRD can be achieved by administering the vaccine composition by the methods and dosage schedules as described herein, and therefore, methods of preventing relapse of multiple myeloma in these patients are possible.

The reasons these methods can be achieved, and further indicators of their success is described herein.

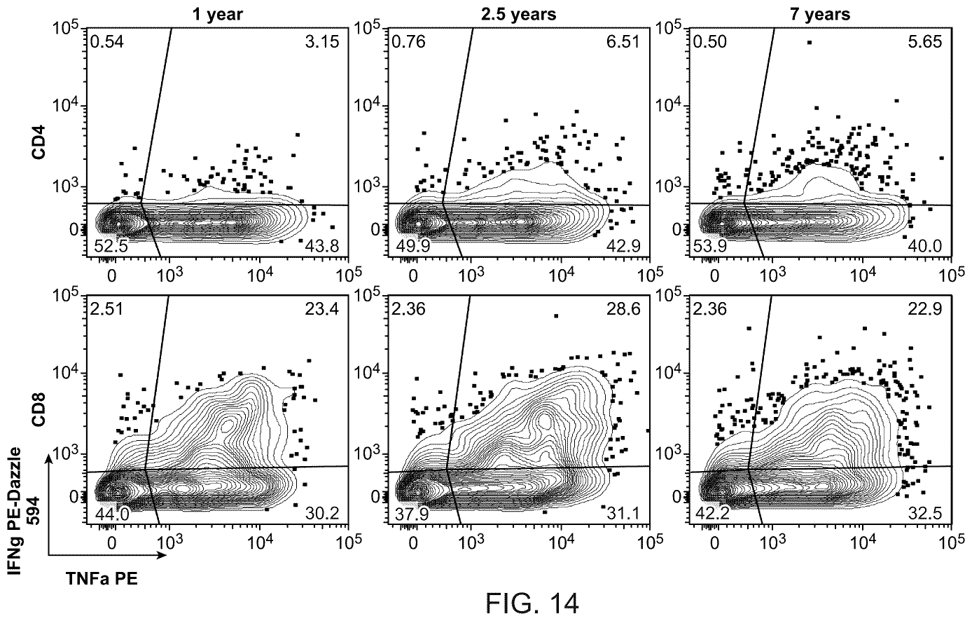

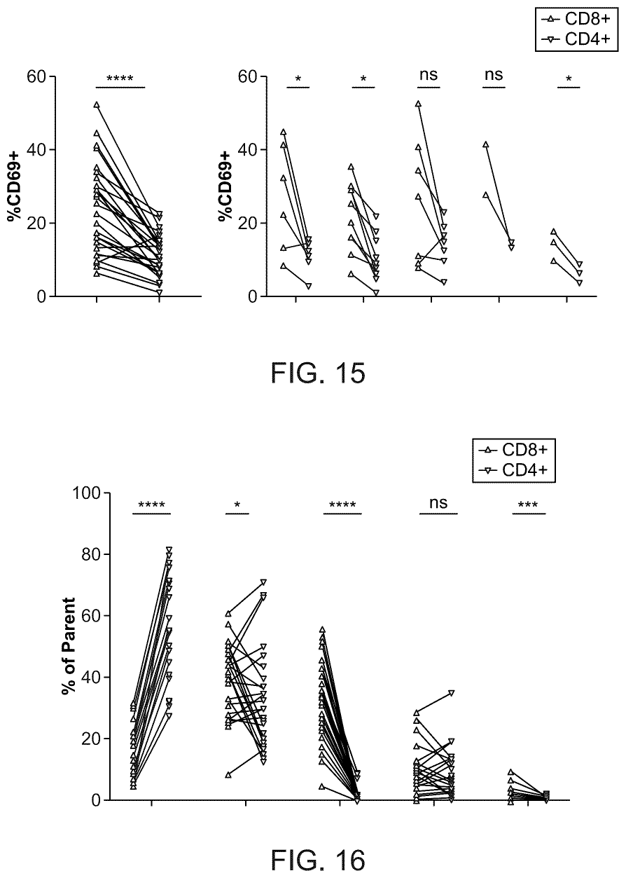

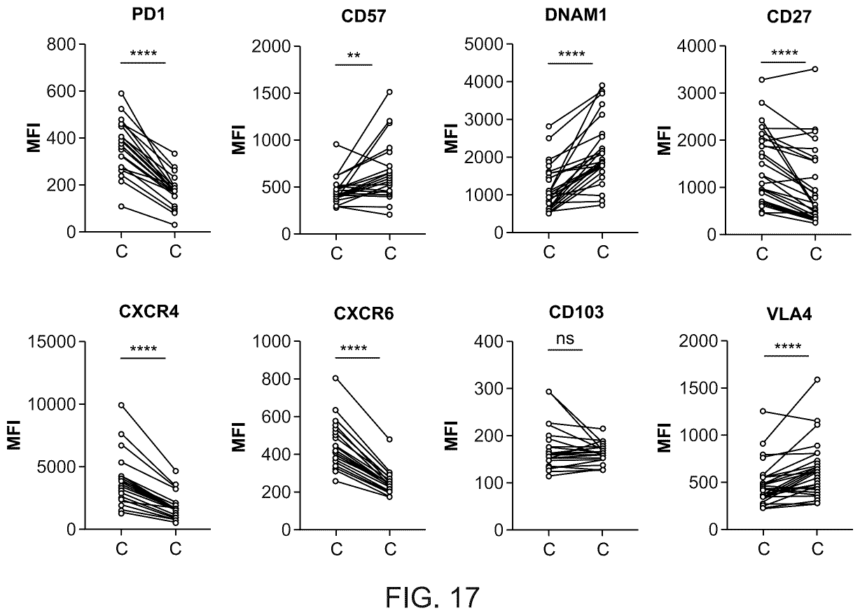

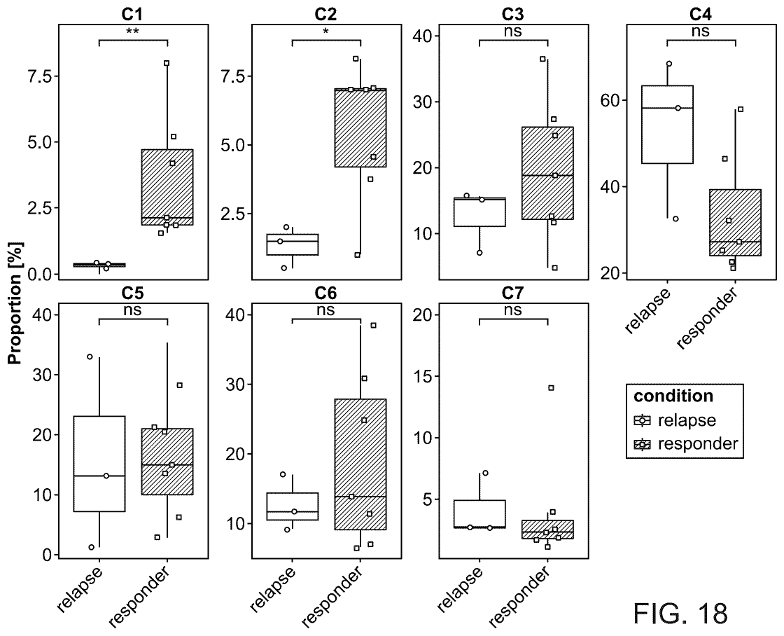

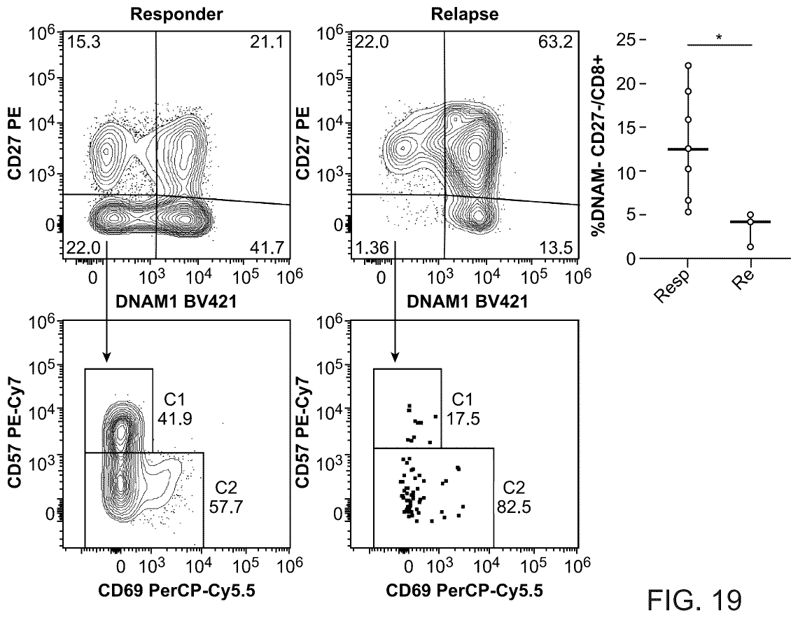

Hence, described herein is evidence of the existence of a bone marrow (tissue)-resident, quiescent T cell population that lacks the hallmarks of exhaustion and senescence and displays high PD1 levels and an effector memory-like phenotype. These observations support the concept of the BM as a reservoir for antigen-experienced memory T cells and provide evidence for the putative mechanism whereby this occurs. Unsupervised clustering of the post-vaccination bone marrow CD8⁺ T cells as described herein enables identification of a subpopulation preferentially enriched in patients with long-term disease remission. This population of DNAM1^{low} CD27⁻ CD8⁺ T cells was virtually absent in the bone marrow of patients with early post-vaccine relapse. Accordingly, CD27 CD8⁺ T cells with a heterogeneous, partially dysfunctional phenotype, defined by the combined expression of both exhaustion and activation markers, are identified as a source of MM-reactive lymphocytes. Their abundance as induced by the vaccine composition as described herein represents a positive prognostic significance in newly diagnosed multiple myeloma patients. On the basis of the MM-MGUS model of persistent immune surveillance, the loss of tumor-reactive CD8⁺ T cell subpopulations would significantly contribute to immune escape and clinically meaningful disease progression. The evidence as presented herein clearly demonstrates that the loss of a potentially tumor reactive CD8⁺ T cell subpopulation preceded clinically evident disease relapse while its persistence correlated with long-term disease remission (FIGS. 14 and 15).

Furthermore, the evidence as presented herein supports the conclusion that the mechanisms whereby vaccination imparts anti-tumor immunity include generating more MM-specific T cells, and also increasing the stem-like, quiescent TRM population within the bone marrow. Moreover, a heterogeneous population of CD8⁺ T cells is identified whose decline precedes clinically evident disease relapse. Phenotypic characterization of the immunophenotypes of BM-resident memory T cells as described herein provide further insight on the important role bone marrow T cells play in the maintenance of MM-specific immunity for several years after vaccination with the vaccine composition as described herein.

The data as described herein fully supports a direct correlation between the depth of MRD response and clinical outcomes. The stable reappearance of the monoclonal protein without meeting the criteria of disease progression as described herein provides evidence that the vaccine composition as described herein is effective in controlling progression and treating multiple myeloma by both increasing the clinical response and/or establishing the MM-MGUS equilibrium that significantly delays disease progression when administered in a low disease burden state.

Current therapies for MM involving IMiDs, proteasome inhibitors and autologous BM transplant with high dose melphalan have dramatically increased the number of patients who achieve a deep clinical response. Multiple myeloma vaccination as described herein in combination with lenalidomide is shown to be an effective treatment to prevent disease relapse in patients who did not achieve complete eradication of MM clones or are known to have short-lived response durations even after achieving MRD negativity.

### EXAMPLES

The embodiments and descriptions as described herein will be more precisely explained below with reference to the following non-limiting examples.

### Example 1: Patient Selection and Eligibility

Eligible patients were at least 18 years old with a diagnosis of multiple myeloma and an Eastern Cooperative Oncology Group (ECOG) performance status of 0-2 with adequate hematopoietic, hepatic and kidney function. Patients were eligible regardless of the number of prior lines of therapy. An autologous hematopoietic stem cell transplant could not have occurred within the past 12 months and prior allogeneic bone marrow transplant was not permitted. To be enrolled, patients had to maintain a sustained near complete remission for an observation period of at least 4 months on a Len-containing regimen. Key exclusion criteria were disease progression after steroid discontinuation, defined as a detectable M-spike >0.5 g/dL or conversion to a true complete remission (defined as absent M-spike and negative serum/urine immunofixation) during the observation period.

Fifteen patients with multiple myeloma (MM) on a Len-containing regimen that achieved a stable near complete remission (nCR) for at least 4 months were enrolled in the trial. A nCR was defined as a non-detectable M-spike and a positive serum and/or urine immunofixation (IFE) (BladÉ et al. Br J Haematol 1998; 102:1115-23). At diagnosis, 53% of the patients had stage I (International Staging System, ISS) disease (Palumbo et al. J Clin Oncol 2015; 33:2863-9). Notably, none of the enrolled patients had high-risk MM features as defined by the IMWG (International Myeloma Working Group) cytogenetic criteria.

At the time of enrollment, Len was continued but all other anti-MM therapy was discontinued. The patients received four MM-GVAX vaccinations at 1, 2, 3, and 6 months in combination with Len at their current dose (FIG. 1) with a median dose of 15 mg (range 2.5-25 mg) for at least 1 year. The median age at enrollment was 69 years (range 45-81 years).

### Example 2: Vaccine Efficacy

All 15 patients were available for clinical response evaluation at 1 year after administration of the first MM-GVAX dose. Eight of 15 patients (53.3%) achieved a disease response consistent with true complete remission (CR), defined as an absent M-spike and negative IFE, with a median time to achieving a CR of 11.6 months (range 1.4-13.9 months) from enrollment. This rate of conversion to CR of 53.3% (95% CI: 26.6-78.7%) was significantly higher than a null hypothesis of 25% conversion to CR rate defined by design (p=0.011) thereby indicating clinical activity and meeting the primary endpoint of this trial. Strikingly, only 6 of the 15 enrolled patients experienced disease progression, defined as appearance of a M-spike of at least 0.5 g/dL and/or an increase in the involved free light chain of more than 10 mg/dL confirmed with a repeat measurement. Pt 6, Pt 7 and Pt 9 experienced early disease relapse within the first year of trial enrollment. Seven patients (46.7%) subsequently developed a detectable M-spike that did not meet the criteria for disease relapse. It was variably persistent over time but did not require any change in treatment. At the time of the analysis, the estimated median overall survival (OS) was 11.5 years from the MM diagnosis (95% CI: 5.9-n/a years) and 7.8 years from enrollment (95% CI: 4.2-7.8 years, n=6/15, 40%). A subgroup analysis of patients achieving a CR showed no statistically significant difference in terms of PFS and OS compared to patients who maintained a stable nCR or those that subsequently developed a measurable M-spike. This finding suggests that vaccination can induce an immune equilibrium capable of maintaining long-term disease control even without the complete eradication of malignant MM plasma cell clones.

### Example 3: Vaccine Formulation and Administration

The cell lines used for vaccine formulation were manufactured by the GMP-compliant Cell Processing and Gene Therapy Facility at the Sidney Kimmel Comprehensive Cancer Center at Johns Hopkins. U266 and H929 were originally acquired from ATCC.

The lentiviral vector is constructed as shown in FIG. 1. The GM-CSF gene is ligated to an EF1a promoter (See FIG. 3) and the resulting DNA construct is flanked on either end with a LTR. The LTRs have identical sequences.

Briefly, the K562 cell line is transduced with the lentiviral vector constructed as described above to create a cell line that is able to express and produce GM-CSF (K562-GMCSF). In brief the K652 cell line is grown in a closed system (Wave Xuri) under conditions of perfusion until adequate numbers for transduction are produced. The cells are then transduced within the culture bag by removing media, adding the lentiviral vector construct for 24 hours and then washing the culture utilizing a closed system. The cells are grown again in perfusion in the Xuri. The cells are then tested for GM-CSF production per a fixed total number of cells. This data is validated by simultaneously running three assays with the same number of cells at several dilutions to determine the total amount of GM-CSF produced every 24 hours.

The cell line is also irradiated and the GMCSF production assay is repeated. The cell line will be administered in the final formulation of the vaccine in an irradiated form.

Equal numbers (5×10⁷ each) of the MM cell lines U266 and H929 were combined with 5×10⁶ cells of the bystander cell line K562GM-CSF. Vaccine cells were irradiated prior to cryopreservation and stored in liquid nitrogen until the day of use. On the day of vaccination, the individual cells were thawed, mixed at the appropriate concentrations and drawn up into three syringes. The final vaccine syringes were kept on ice until administration that occurred within 60 minutes after thawing.

Patients received four vaccinations on day 14 of the 1st, 2nd, 3rd and 6th month from enrollment (cycles 1, 2, 3, and 6, respectively), while continuing Len for at least a year at the dose administered prior to enrollment. Len was administered for 21 out of 28 days. On a single vaccination encounter, patients received intra-dermal MM-GVAX administered over three limbs in a total volume not greater than 1 mL and the pneumococcal conjugate vaccine 13 (PCV-13, Prevnar®) injected intramuscularly in one arm. Upon completion of the study, patients continued treatment with Len assuming evidence of clinical efficacy. All subjects underwent safety assessments, disease response determinations and sampling of BM and PB for correlative biomarker analysis just prior to treatment (baseline), at day 14 of cycle 3 (C3D14, prior to the third vaccine dose) and 1 year after enrollment. For some patients, additional follow-up BM and PB samples were collected past the 1-year timepoint and up to seven years after trial enrollment.

### Example 4: MRD Burden Enables Prediction of Vaccine Response

To quantify and track the MM disease burden, minimal residual disease (MRD) testing by next generation sequencing was performed on 7 (46.7%) patients for whom baseline MM DNA was available (Rawstron et al. “A complementary role of multiparameter flow cytometry and high-throughput sequencing for minimal residual disease detection in chronic lymphocytic leukemia: an European Research Initiative on CLL study”; Leukemia [Internet]. 2016; 30:929-36. Available from: nature.com/articles/leu2015313). Since the tested samples in all but one subject had an MRD frequency greater than 10⁻⁶ (10⁻⁴%) in the B-cell repertoire, an arbitrary threshold of 10⁻³ (10⁻¹%) was identified that allowed for evaluation of the clinical significance of a disease burden above (high-level MRD⁺) and below (low-level MRD⁺) this threshold in the bone marrow.

Dominant IGH and IGK/L cancer clones were identified from immunosequencing results in pre-treatment bone marrow using the following criteria: 1) The sequence must have frequency >5%; 2) The sequence must be present at >0.1% of the total nucleated cells; 3) The sequence must be discontinuously distributed (four or fewer sequences in the next decade of sequence frequencies); 4) The sample must have a template estimate of >200. These identified dominant clones were tracked over time in bone marrow to determine the frequency of the cancer clone(s) at subsequent time points after treatment. To account for somatic hypermutation (SHM), IGH clones that had 2 or fewer mismatches with the dominant clone were also tracked in bone marrow over time. The MRD frequency in each sample was measured as the frequency of the cancer clones among all productive rearrangements of the locus being tested.

All high-level MRD⁺ patients experienced disease relapse within a year from enrollment (median=4.8 months, range: 2.8-9.5 months), while low-level MRD⁺ patients had a significantly longer PFS (median=84.15 months, range: 51.9-97.3 months, p=0.01). Detection of high-level MRD compared with low-level MRD was associated with an increased likelihood of clinical relapse (hazard ratio, HR=25.79, 95% CI: 2.17-306.4). Notably, Pt 2 and Pt 4 had a prolonged clinical response despite developing a variably detectable M-spike that never met the criteria for disease progression. In contrast, Pt 12 and Pt 13 achieved a true CR and maintained their response over time. Therefore, MRD burden at the time of vaccination is predictive of long-term clinical outcomes.

### Example 5: MM-GVAX Vaccination Induces Systemic Myeloma Immunity

To determine the impact of MM-GVAX vaccination on the global T cell receptor (TCR) repertoire, we performed deep sequencing analysis of the TCR chain Vbeta (TRBV) on matched peripheral blood (PB) and bone marrow (BM) samples from all patients at baseline, prior to the third MM-GVAX dose (C3D14) and at 1 year. Immunosequencing of the CDR3 regions of human TCRβ and IGH and IGK/L chains from T cells and B cells, respectively, was performed on genomic DNA extracted from BM or PB samples using the immunoSEQ® Assay (Adaptive Biotechnologies, Seattle, WA). Extracted genomic DNA was amplified in a bias-controlled multiplex PCR, followed by high-throughput sequencing. Sequences were collapsed and filtered in order to identify and quantitate the absolute abundance of each unique CDR3 region for further analysis as previously described (Robins et al. “Comprehensive assessment of T-cell receptor β-chain diversity in αβ T cells”; Blood [Internet]. 2009; 114:4099-107. Available from ashpublications.org/blood/article/114/19/4099/26771; Carlson et al. “Using synthetic templates to design an unbiased multiplex PCR assay”; Nat Commun [Internet]. 2013; 4:2680. Available from: nature.com/articles/ncomms3680; Robins et al. “Ultra-sensitive detection of rare T cell clones”; J Immunol Methods [Internet]. 2012; 375:14-9. Available from:
linkinghub.elsevier.com/retrieve/pii/S0022175911002468; Kirsch et al. “T-cell receptor profiling in cancer; Mol Oncol [Internet]. 2015; 9:2063-70. Available from:
doi.wiley.com/10.1016/j.molonc.2015.09.003; DeWitt et al. “A Public Database of Memory and Naive B-Cell Receptor Sequences”; Turner SJ, editor. PLOS One [Internet]. 2016; 11: e0160853. Available from: dx.plos.org/10.1371/journal.pone.0160853). Only productive rearrangements were used for repertoire analysis.

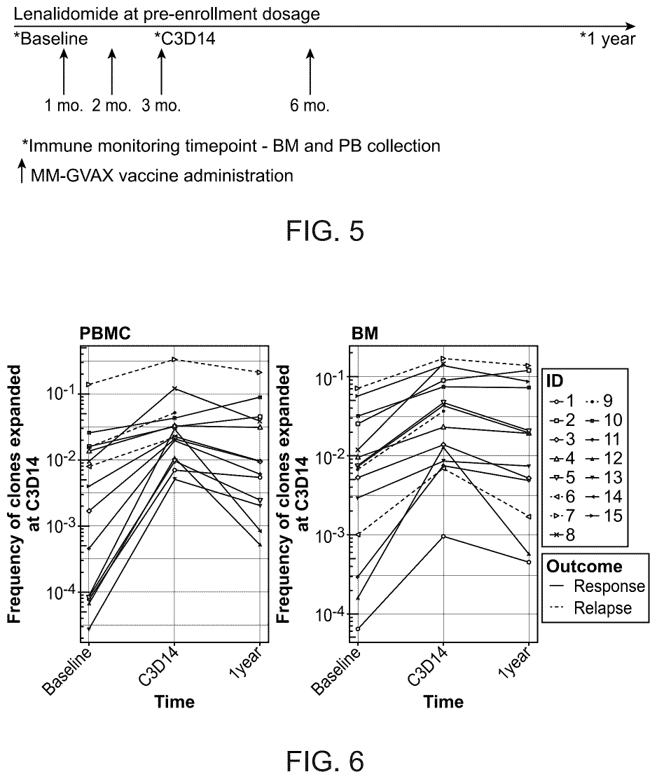

Prior to vaccination, the TCR clonotypic composition was varied and ranged from minimal repertoire bias to significant oligoclonal expansion. Despite the hypothesis that vaccination should skew the TCR repertoire towards increased clonality, we did not observe major changes in the relative proportions of productive TCR rearrangements in either compartment. Productive clonality varied greatly among different patients and over time, but no vaccine-related pattern could be identified. Overall, productive clonality appeared to be relatively stable over time in most subjects and did not correlate with clinical outcomes. Considering that minimal TCR repertoire skewing was observed with vaccination, we examined the changes in clonal abundance pre- and post-vaccination by comparing the frequencies of each clone. Notably, there were significantly expanded T-cell clones in both the PB and BM in all subjects after the first two MM-GVAX doses (at C3D14), and these clones persisted at the high frequency reached after their initial expansion following vaccination (FIG. 2). Despite the absence of changes in overall clonality associated with vaccination, there is clear evidence of clonal expansion and contraction in both PB and BM samples. Interestingly, many of the significantly expanded clones after vaccination were not observed prior to vaccination suggesting either the expansion of previously undetected clones or the recruitment of novel clonotypes upon vaccination (FIG. 3). The expansion of T cell clones in both the PB and BM underscores the ability of MM-GVAX to induce a broad systemic immune response. Although most clones were present at comparable frequencies in PB and BM, some clonotypes were preferentially enriched in the BM (FIG. 4). To better understand the clonal dynamics associated with vaccination, the Morisita index was used to determine the similarity between TCR repertoires in these two compartments. In some patients (Pt 7, Pt 10 and Pt 15), the BM and PB TCR repertoires were more disparate from each other after vaccination compared to baseline, whereas in most subjects there was little change in the similarity between the two compartments after vaccination (FIG. 5). Further analysis of expanded BM and PB T cell clones after vaccination revealed diverse clonal expansion behaviors across patients. In some, expanded clonotypes increased in frequency in both PB and BM, suggesting parallel processes in both compartments. Conversely, in others, some clones expanded in one compartment but not the other or expanded in both compartments but displayed preferential enrichment in either the BM or PB. Collectively, these results show that MM-GVAX induced a systemic T-cell response with measurable clonal expansion in both BM and PB.

### Example 6: Vaccination Induces MM-Specific Polyfunctional T Cell Responses in the Bone Marrow

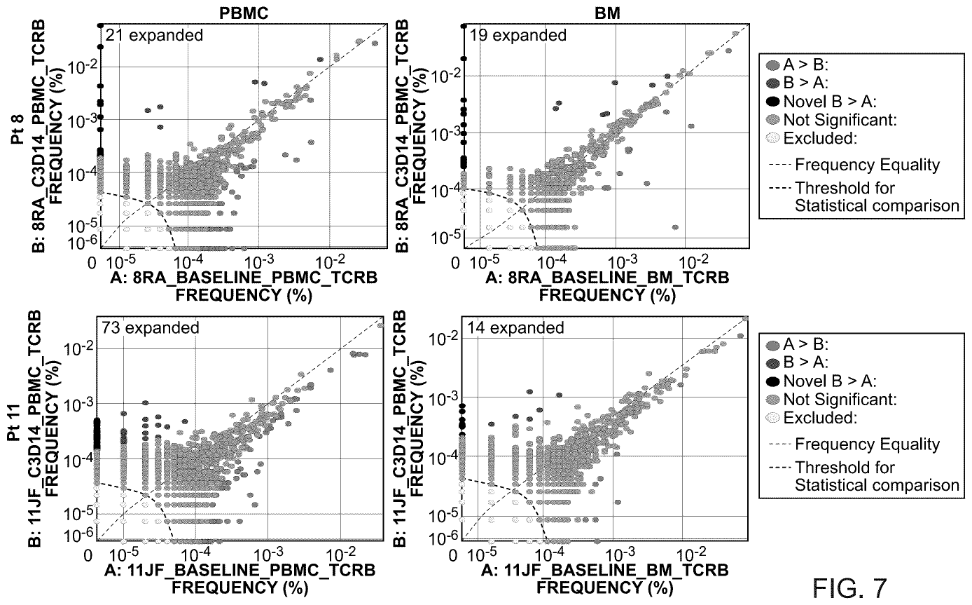

T cell responses were functionally characterized to both vaccine-related and unrelated MM antigens in the BM. Samples from all patients and timepoints were stimulated in vitro with lysates from the MM-GVAX cell lines (U266 and H929) and analyzed for intracellular cytokine production. MM-GVAX-specific interferon-γ (IFNγ) and TNFα responses markedly increased upon vaccination in both CD8⁺ and CD4⁺ T cell subsets at C3D14 and 1 year (FIG. 6). The frequency of CD4⁺ or CD8⁺ T cells producing either IFNγ and/or TNFα in response to vaccine-related and unrelated MM-antigens significantly increased with only two vaccinations and remained persistently elevated for up to 4 years or more (p<0.0001, FIG. 7). BM-derived mononuclear cells obtained at the indicated timepoints before and after vaccination were stimulated either in AIM-V medium with 2% human AB serum alone or with SW780 (bladder carcinoma cell line) lysate or with U266/H929 (MM-GVAX cell lines) lysates, respectively. After 5 days, cells were harvested and stained for flow cytometric analysis of intracellular cytokine production.

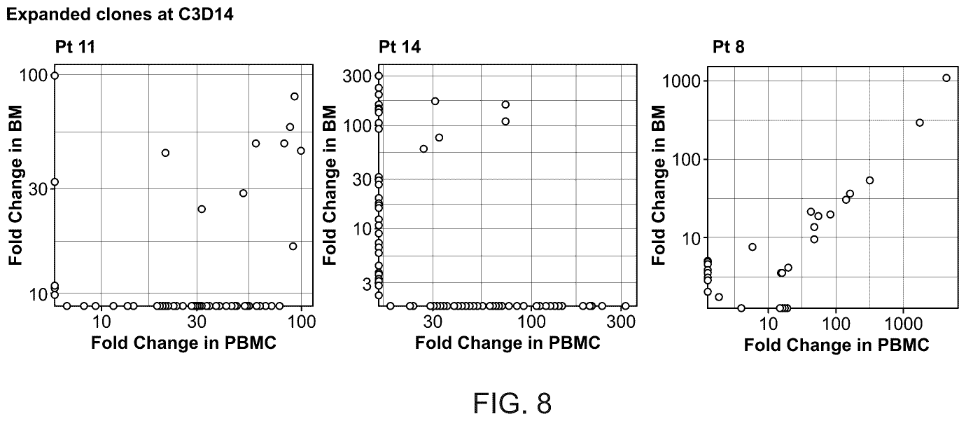

Notably, MM-GVAX significantly increased the frequency of polyfunctional CD4⁺ and CD8⁺ T cells, defined as co-producing IFNγ and TNFα, as well as the fraction of single-cytokine producing T cells, albeit to a lower extent. Interestingly, the most profound changes appeared to be the generation of CD8⁺ T cells producing either TNFαor IFNγ/TNFα(FIG. 8). Pt 6, Pt 7 and Pt 9, who relapsed early after vaccination, developed vaccine-specific T cell cytokine responses comparable to patients that achieved long-term disease remission. These results demonstrate that the MM-GVAX-induced immune response is polyfunctional, directed towards a broad range of commonly shared MM-associated antigens and involves both CD8⁺ and CD4⁺ T cells. These findings support data from other studies showing that effective vaccine-induced anti-tumor immunity requires both CD8+ and CD4⁺ tumor-specific T cells (Bennett et al. “Induction of a CD8⁺+Cytotoxic T Lymphocyte Response by Cross-priming Requires Cognate CD4⁺+ T Cell Help”; J Exp Med [Internet]. 1997; 186:65-70. Available from: rupress.org/jem/article/186/1/65/7193; and Alspach E, Lussier et al.; “MHC-II neoantigens shape tumour immunity and response to immunotherapy”; Nature [Internet]. 2019; 574:696-701. Available from: nature.com/articles/s41586-019-1671-8).

### Example 7: Vaccine-Induced MM-Specific T Cell Immunity Persists for Several Years after Vaccination

Important hallmarks of adaptive immunity are its persistence over time and its capacity to mount an effective response upon antigen re-encounter. As such, MM-GVAX-specific responses in available samples collected several years after vaccination were detected and characterized.

Bone marrow and peripheral blood samples were collected at the pre-established timepoints, enriched for mononuclear cells using Lymphoprep (STEMCELL Technologies®) gradient and cryopreserved in freezing media (50% complete AIM-V media, 40% human decomplemented AB serum and 10% DMSO). Samples were then thawed and washed twice with prewarmed (37C) AIM-V with 0.02 mg/mL DNase and phosphate buffered saline (PBS), respectively. Flow cytometry reagents were purchased from BioLegend, BD Biosciences and Invitrogen. Monoclonal antibodies were previously titrated to the optimal concentration. Surface staining was performed for 20 minutes at 37C, while intracellular detection of cytokines was performed following fixation of cells with CytoFix/CytoPerm kit (BD Biosciences) according to the manufacturer's instructions and by incubating the cells with specific mAb cocktails for 20 minutes at room temperature. All data were acquired on a Gallios® Flow Cytometer (Beckmann-Coulter) equipped with three lasers (violet, 405 nm; blue, 488 nm; red, 633 nm) and capable of detecting 10 parameters. Flow cytometry data were compensated in FlowJo by using single stained cell controls and compensation beads (BioLegend). After pre-processing by biexponential transformation, standard gating to remove aggregates and dead cells, CD3+CD8⁺ T cells were subsequently exported from FlowJo for further analysis in R (version 4.0.1) by a custom-made script that used Bioconductor libraries and R packages. Briefly, data were analyzed using the FlowSOM algorithm for unsupervised clustering and visualized with UMAP. Differential discovery analyses were performed on R using the diffcyt framework and the CATALYST workflow (Nowicka et al.; “CyTOF workflow: differential discovery in high-throughput high-dimensional cytometry datasets”; F1000Research [Internet]. 2019; 6:748. Available from: f1000research.com/articles/6-748/v3). Data were then reorganized as new files, one per each cluster and further analyzed in FlowJo to determine the frequency of positive cells for each marker and their mean fluorescent intensity (MFI).

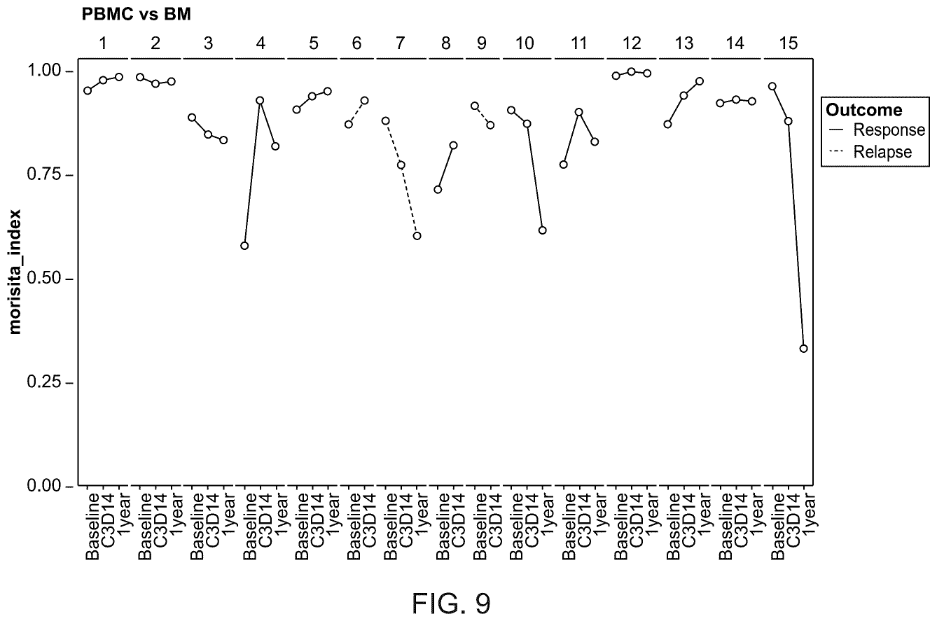

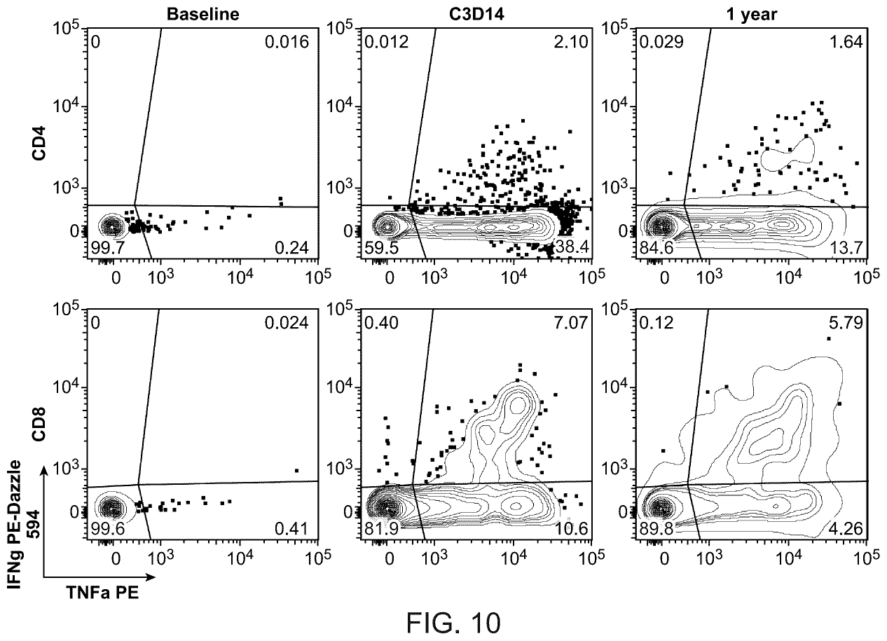

In three patients for whom long-term samples were available, expanded clonotypes detected at C3D14 were still present up to seven years post-vaccination. Surprisingly, the frequency of expanded clones in each compartment remained relatively stable in both BM and PB from 1-year post-vaccination onward, suggesting the establishment of a steady-state immune-equilibrium (FIG. 9). Intracellular cytokine staining of these BM samples stimulated with both MM-GVAX-related and unrelated MM-antigens showed a persistent polyfunctional CD4⁺ and CD8⁺ T cell response up to 7 years post-vaccination (FIG. 10). In summary, the persistence of vaccine-specific clones in PB and BM as well as of MM-specific polyfunctional T cells for several years after vaccination even in the presence of detectable disease suggests the establishment of an immune equilibrium that is likely responsible for the long-term disease control observed in this study.

### Example 8: T Cells in the BM Display an Effector Phenotype and a Tissue Resident-Like Signature

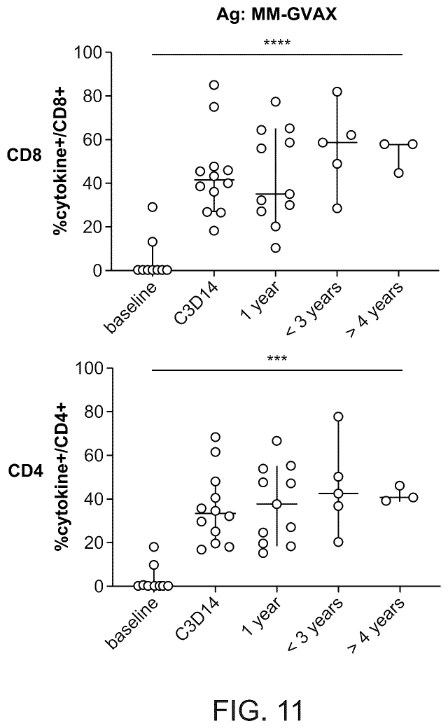

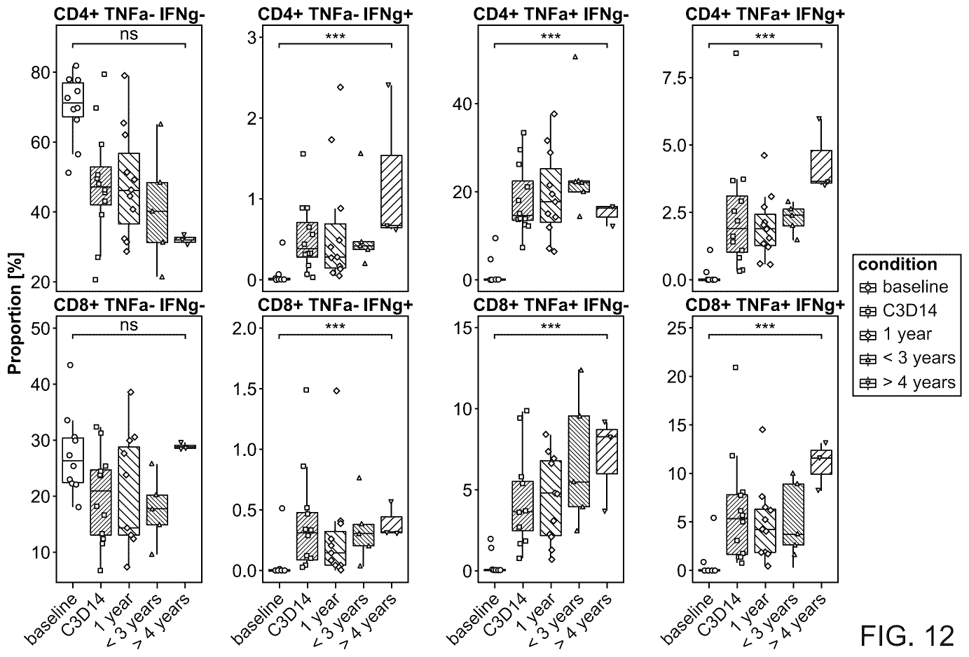

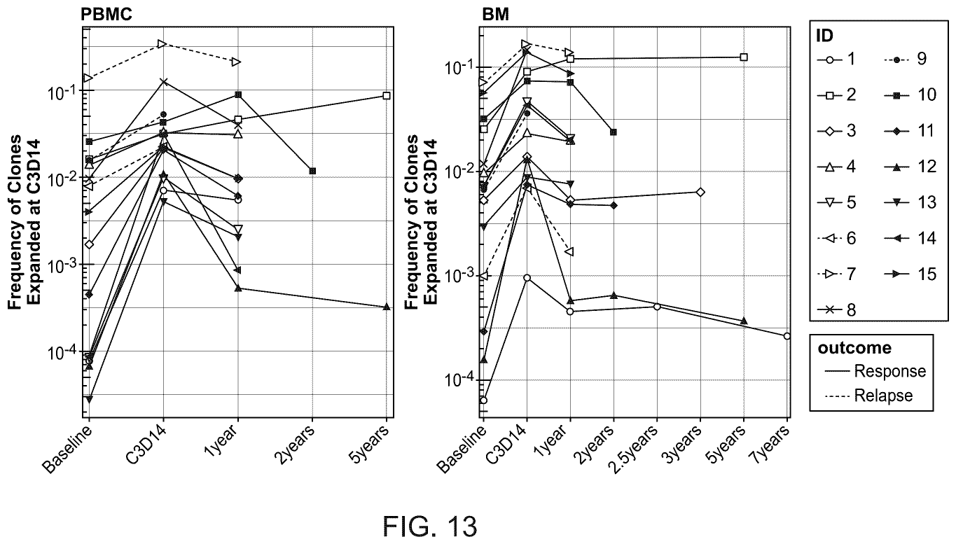

The phenotypic composition of BM T cells was examined for their expression of checkpoint molecules, costimulatory molecules and chemokine receptors. The BM T cell composition was remarkably similar across timepoints (data not shown). A CD69-expressing, tissue resident-like T cell population (T_{RM}) was identified that was consistently present in all BM samples. The proportion of CD69⁺ T_{RM} was mostly unvaried over time, but CD8⁺ T_{RM} were more prevalent than their CD4⁺ counterparts (FIG. 11). Canonical phenotypic identification of T cell subsets revealed that a higher proportion of CD4⁺ T cells displayed a central memory phenotype (T_{CM}), while effector memory (T_{EM}) and effector (TEMRA) subsets were more represented among CD8⁺ T cells. Interestingly, stem cell memory-like T cells (T_{SCM}) were present at a higher frequency among CD8⁺ T cells compared to the CD4⁺ subset (FIG. 12). BM TRM mainly exhibited T_{EM} and TEMRA phenotypes, although TCM- and TSCM-like TRMs could be detected to a lesser extent. CD69⁺CD8⁺ T cells in the BM represent a memory population with hallmarks of tissue residency. Interestingly, they lack markers of exhaustion, such as TIGIT and TIM3, and senescence (CD57) but express the activation markers CD27 and PD1. Interestingly, BM TRMs expressed higher levels of both CXCR4, a BM homing chemokine receptor, and CXCR6, which is considered a hallmark of tissue-resident T cells (Kumar et al., “Human Tissue-Resident Memory T Cells Are Defined by Core Transcriptional and Functional Signatures in Lymphoid and Mucosal Sites”; Cell Rep [Internet]. ElsevierCompany.; 2017; 20:2921-34. Available from:
dx.doi.org/10.1016/j.celrep.2017.08.078.) (FIG. 13). A significant percentage of patients maintained disease control albeit with the reappearance of low levels of monoclonal protein that persisted over time, but did not meet the criteria of relapse. This stable, low-level disease is suggestive of the establishment of a state of immune equilibrium. The identification of a BM resident T cell population that is consistently present in this cohort supports previous findings that TRMS have a fundamental role in maintaining an immune equilibrium and in tumor surveillance (Park et al., “Tissue-resident memory CD8⁺+ T cells promote melanoma-immune equilibrium in skin”; Nature [Internet]. 2019; 565:366-71. Available from: nature.com/articles/s41586-018-0812-9).

### Example 9: Immune Correlates of Clinical Outcome after MM-GVAX Vaccination

To gain insight into the immunophenotypes of BM CD8⁺ T cells post-vaccination and their association with clinical outcome, C3D14 BM CD8⁺ T cells were analyzed with FlowSOM, an unsupervised clustering algorithm, and used dimensionality reduction approaches, such as Uniform Manifold Approximation and Projection (UMAP), to simplify the visualization of different T cell clusters. Hierarchical metaclustering of FlowSOM clusters grouped CD8⁺ T cell subpopulations with similar immunophenotypes and identified two subsets of CD8⁺ T cells, i.e., C1 and C2, that were enriched in patients with durable disease control (responder group) compared to those showing early progressive disease (relapse group) within the first year after vaccination (FIG. 14, *, p<0.05; **, p<0.01). Remarkably, clusters C1 and C2 were defined by low-to-absent DNAM1 expression and lack of CD27, and included relatively heterogeneous subpopulations including senescent, effector and exhausted CD8⁺ T cells. Further characterization of cluster C2 identified a CD69+CD57-subpopulation with intermediate PD1 expression, suggesting that these CD8⁺ T cells enriched in the responder group are BM-resident and likely involved in long-term MM control. Interestingly, cluster C1 was characterized by increased CD57 expression, suggesting that these effector-senescent cells may still be functional, despite their lack of proliferative potential. The results obtained with the FlowSOM algorithm were subsequently reproduced by standard flow cytometry approaches where manually gated CD27-DNAMIlow/−CD8⁺ T cells were enriched in vaccine-responders (FIG. 15). These findings provide evidence that changes in the CD8⁺ T cell composition occur earlier than clinically evident disease relapse and identify a subset of BM CD8⁺ T cells that may be a reservoir of tumor-specific T cells and likely plays a role in the establishment and maintenance of the vaccine-induced immune equilibrium.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A lentiviral vector construct comprising a DNA sequence encoding GM-CSF.

2. The lentiviral vector construct of claim 1, further comprising a DNA sequence encoding EF1a.

3. The lentiviral vector construct of claim 2, wherein the DNA sequence encoding EF1a is shown in SEQ ID NO: 2.

4. The lentiviral vector construct of claim 1, wherein an amino acid sequence of GM-CSF is shown in SEQ ID NO: 3.

5. The lentiviral vector construct of claim 1, comprising the structure LTR-EF1a-GMCSF-LTR.

6. The lentiviral vector construct of claim 5, wherein DNA encoding the LTR is shown in SEQ ID NO: 1.

7. A composition comprising K562 cells, wherein the K562 cells comprise the vector construct of claim 1.

8. The composition of claim 7, wherein the composition is a vaccine.

9. The composition of claim 8, wherein said vaccine is allogeneic.

10. The composition of claim 1, further comprising U266 and H929 cells.

11. The composition of claim 7, wherein the DNA sequence encoding GM-CSF is able to produce GM-CSF in an amount of up to about 1500 ng/1×10⁶ cells.

12. The composition of claim 11, wherein the amount of GM-CSF produced is between about 35-1200 ng/1×10⁶ cells.

13. The composition of claim 7, wherein the GM-CSF is derived from human.

14. The composition of claim 10, wherein the ratio of the combination of U266 and H929 cells to K562 cells is about 20:1.

15. The composition of claim 10, wherein the U266 and H929 cells are present in equal amounts.

16. The composition of claim 10, wherein the U266 and H929 cells are present in unequal amounts.

17. The composition of claim 7, wherein said composition induces an immune response in a subject when administered to said subject.

18. The composition of claim 17, wherein the immune response induces complete remission of said plasma cell disorder in the subject.

19. The composition of claim 17, wherein the composition prolongs progression free survival in said subject.

20. The composition of claim 17, wherein said complete remission is determined as a non-detectable M-spike and positive immunofixation electrophoresis.

21. The composition of claim 17, wherein the subject is a human.

22. A method of treating a plasma cell disorder in a subject, comprising administering to the subject the composition of claim 7.

23. The method of claim 22, wherein said administering comprises also administering lenalidomide to said subject.

24. The method of claim 24, wherein said lenalidomide is administered to said subject before, during, and/or after said administering.

25. The method of claim 22, wherein the composition is a vaccine.

26. The method of claim 25, wherein the vaccine is allogeneic.

27. The method of claim 22, wherein the K562 cells express a GM-CSF gene.

28. The method of claim 22, wherein the K562 cells have been transfected with a vector construct encoding GM-CSF.

29. The method of claim 28, wherein the GM-CSF gene is able to express an amount of GM-CSF of up to about 1500 ng/1×10⁶ cells.

30. The method of claim 29, wherein the amount of GM-CSF expressed is between about 35-1200 ng/1×10⁶ cells.

31. The method of claim 29, wherein the amount of GM-CSF is produced, on average, every 24 hours.

32. The method of claim 22, wherein the GM-CSF is derived from human.

33. A method of treating a plasma cell disorder in a subject, comprising administering to the subject the composition of claim 10.

34. The method of claim 28, wherein the ratio of the combination of U266 and H929 cells to K562 cells is about 20:1.

35. The method of claim 28, wherein the dose of said composition is such that the ratio of tumor cells in said subject to K562 cells in said composition is greater than 2:1.

36. The method of claim 28, wherein the U266 and H929 cells are present in equal amounts in said composition.

37. The method of claim 28, wherein said U266 and H929 cells are present in said composition in an amount of about 5×10⁷ cells and the K562 cells are present in said composition in an amount of about 5×10⁶ cells.

38. The method of claim 28, wherein near or complete remission is achieved in said subject.

39. The method of claim 28, wherein said near or complete remission persists in said subject for up to 5 years.

40. The method of claim 33, wherein said complete remission is determined by measuring no detectable monoclonal spike and negative immunofixation electrophoresis.

41. The method of claim 28, wherein said subject is positive for minimal residual disease.

42. The method of claim 28, wherein said composition minimizes a non-specific immune response in the subject.

43. The method of claim 28, wherein said composition is administered to said subject in 1 to 5 doses, spaced apart by more than 1 day between each dose.

44. The method of claim 38, wherein 2 to 4 doses are administered, spaced apart by more than 2 weeks between each dose.

45. The method of claim 38, wherein there are more than 4 weeks between each dose.

46. The method of claim 38, wherein 4 doses are administered, spaced apart by about 1 month between each dose.

47. The method of claim 38, wherein the first 3 doses are spaced apart equidistantly.

48. The method of claim 38, wherein all doses are administered within one year relative to each other.

49. The method of claim 38, wherein at least one dose is administered between and including days 7-18 relative to starting a course of lenalidomide.

50. The method of claim 38, wherein at least one dose is administered on about day 15 relative to starting a course of lenalidomide.

51. The method of claim 38, wherein said plasma cell disorder is selected from the group consisting of MGUS, SMM, multiple myeloma, non-secretory multiple myeloma, indolent myeloma, light chain myeloma, plasma cell leukemia, and primary amyloidosis.

52. The method of claim 38, wherein said plasma cell disorder is multiple myeloma.

53. A method of prolonging progression free survival in a subject having multiple myeloma comprising administering the composition of claim 10 to said subject in combination with lenalidomide.

54. A method of inducing an increase in clonal T-cell expansion and a myeloma-specific cytokine response in a subject having multiple myeloma comprising administering to the subject the composition of claim 10 in combination with lenalidomide.

55. The method of claim 49, wherein said increase persists in said subject for up to 7 years after said administering.

56. The method of claim 49, wherein said increase persists in said subject for up to 5 years after said administering.

57. A method of inducing multiple-myeloma-specific immunity in a subject comprising administering to the subject the composition of claim 10 in combination with lenalidomide.

58. The method of claim 52, wherein said subject is positive for minimal residual disease at the time of said administering.

59. A method of preventing relapse of multiple myeloma in a subject, comprising administering to the subject the composition of claim 10 in combination with lenalidomide.

60. The method of claim 54, wherein the subject is positive for minimal residual disease at the time of said administering.

61. The method of claim 22, wherein the subject is a human.


SEQUENCE LISTING (3 sequences)

SEQ ID NO: 1 - DNA, 9202 bp, synthetic construct
  source 1..9202: mol_type = other DNA
tgagctagctctagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggaggcagcggagagggcagaggaagtcttctaacatgcggtgacgtggaggagaatcccggccctaggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcg

SEQ ID NO: 2 - DNA, 1189 bp, synthetic construct
  source 1..1189: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 3 - protein, 127 aa, synthetic construct [sequence omitted]